(19) United States
(12) Patent Application Publication
(10) Pub. No.: US 2003/0135212 A1
(43) Pub. Date: Jul. 17, 2003

(54) Rod and plate bone fixation device for persons with osteophorosis

(76) Inventor: James C. Y. Chow, Mount Vernon, IL (US)

Correspondence Address: POLSTER, LIEDER, WOODRUFF & LUCCHESI, 763 SOUTH NEW BALLAS ROAD, ST. LOUIS, MO, 63141-8750, US

(21) Appl. No.: 10/044,054

(22) Filed: Jan. 11, 2002

(51) Int. Cl. A61B 17/58

(52) U.S. Cl. 606/64

(57) ABSTRACT

A bone fixation device (10) employs a rod (12) inserted longitudinally of a broken bone (B) and bridges a fracture (Fx) in the bone. Screws (14) are inserted transversely of the bone to hold the rod in place. A relatively thin plate (16) is contoured to fit against the bone on opposite thereof. The plates are form fitted against the sides of the bone and the screws are inserted into the bone through spaced openings (18) in the plates. The heads (20) of the screws are drawn up against the side of the plate and washers (22) are used with to distribute the forces exerted on the bone by the head of the screw. The plates prevent the screws from sinking into the bone and creating stresses which might cause displacement of the reduced fracture.

### CROSS REFERENCE TO RELATED APPLICATIONS

None

### STATEMENT REGARDING FEDERALLY SPONSORED RESEARCH OR DEVELOPMENT

Not applicable

### BACKGROUND OF THE INVENTION

This invention relates to the repair of bone fractures in humans in which pins or rods are inserted into the bone with screws and sometimes plates being used to help hold the parts of the repaired bone together. In particular, this invention relates to a bone fixation device and method for use with person's suffering from osteophorosis or the possibility of future loss of bone mass, so prevent to a recurring injury to a repaired bone as the person ages.

Referring to FIG. 1, current repair techniques for fixing bone fractures often include the insertion of a pin or rod R longitudinally through a broken bone B. In FIG. 1, the fracture is indicated Fx and the length of the rod R is such that it extends across the fracture between bone segments B1 and B2. The center portion of the bone comprises a relatively soft marrow tissue M; while at the end of the bone, the entire bone tissue is a soft spongy tissue Y. The rod is inserted into the bone through this tissue. The rod is a solid rod with spaced, transverse openings O in it. To help hold the rod in place, screws S are inserted into the bone and through the openings in the rod both above and below the location of the fracture. With the screws in place, the rod cannot move and the broken portions of the bone are held together. The rods and screws are typically left in place after the fracture heals.

In some surgical procedures a plate is used to help secure the screws to the side of the bone. Various plate and screw devices or systems are currently in use. U.S. Pat. Nos. 6,171,307, 6,010,504, 5,201,735, 5,129,899, 5,108,399, 5,013,314, and 4,964,403 are representative of the state of the art. The plates currently used in bone surgery are relatively thick, being on the order of 5-8 mm thick. This thickness is required for strength.

It is well understood that as some people (particularly women) age, their bones lose mass. In certain instances this can create the problem illustrated in FIG. 2 of the drawings. Here, as a result of osteophorosis, there is less bone tissue to support the screws, particularly near the end of the bone. Now, the screws shown in the lower portion of the drawings sink into the spongy bone tissue Y. This occurs even if, for example, washers (not shown) are placed between the head of the screw and the outer surface of the bone; for while the washer may distribute some force, as the bone softens due to loss of mass, it is not prevented from sinking into the surface of the bone. The resulting lateral displacement produces a stress at the location of the fracture and can cause displacement of the reduced fracture to occur. This often means another surgery to repair the displacement. Or, the patient otherwise has to accept the displacement which is a less desirable result. As the overall population ages, there will be an increase in this problem.

Surgery on older adults often results in longer healing times and the possibility of complications from surgery increases with age. Therefore, it would be beneficial to avoid them if possible. The present invention is designed to address this problem and to provide a solution which prevents the recurrence of fractures such as described above.

### BRIEF SUMMARY OF THE INVENTION

In accordance with the present invention, briefly stated, a rod and plate bone fixation device of the present invention employs a rod inserted into the broken bone of a person. The rod is inserted longitudinally of the bone and bridges a fracture in the bone. Screws are inserted into the bone, transversely of the bone, and through openings in the rod to hold the rod in place. A relatively thin plate is contoured to fit against the outer surface of the bone on opposite sides of the bone. The thinness of the plate enables it to be form fitted against the side of the bone. A series of screws are inserted into the bone through spaced openings in the plate with the head of the screws being drawn up against the side of the plate. A washer is used with each screw to distribute the force exerted on the bone by the head of the screw. As the person ages, even if they lose bone mass, the plate now prevents the screws from sinking into the bone and creating stresses on the bone which might cause a new fracture. Other objects and features will be in part apparent and in part pointed out hereinafter.

### BRIEF DESCRIPTION OF THE SEVERAL VIEWS OF THE DRAWINGS

The objects of the invention are achieved as set forth in the illustrative embodiments shown in the drawings which form a part of the specification.

FIG. 1 is a representation of a prior art technique for repairing bone fractures using rods and screws;

FIG. 2 illustrates a subsequent displaced fracture to the bone resulting from bone loss as the person ages;

FIG. 3 is side elevational view of a bone fracture repaired using the device of the present invention;

FIG. 4 is a front elevational view of a plate used in the device;

FIG. 5 is side elevational view of the device showing the relative thickness of the plate to a plate used in conventional surgeries;

FIG. 6A is top plan view of the plate and FIG. 6B is a bottom plan view thereof; and, FIG. 7 is a perspective view of the plate.

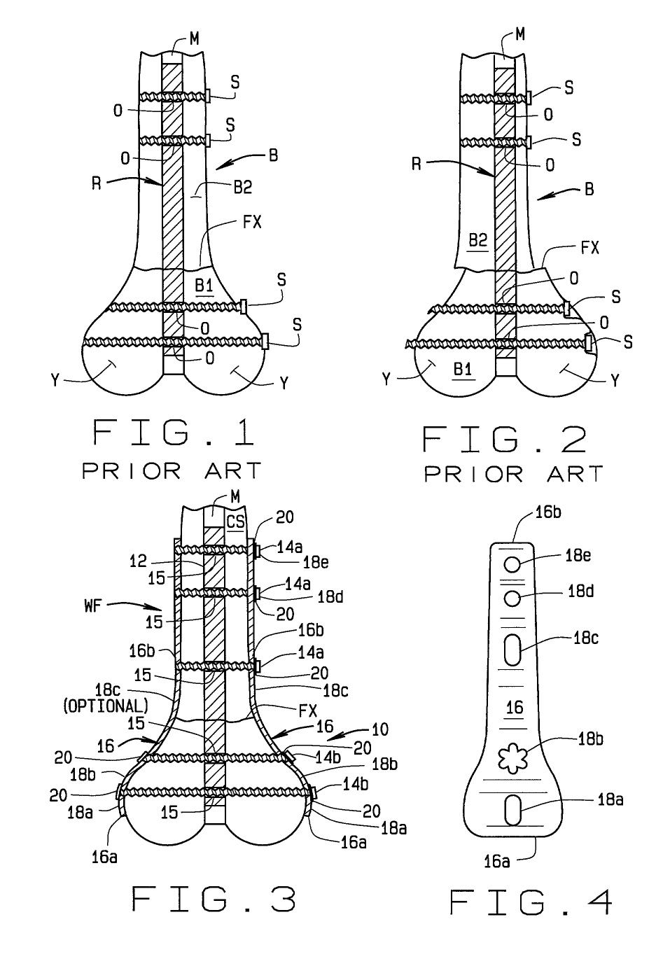

Corresponding reference characters indicate corresponding parts throughout the several views of the drawings.

### DETAILED DESCRIPTION OF INVENTION

The following detailed description illustrates the invention by way of example and not by way of limitation. This description will clearly enable one skilled in the art to make and use the invention, and describes several embodiments, adaptations, variations, alternatives and uses of the invention, including what I presently believe is the best mode of carrying out the invention. As various changes could be made in the above constructions without departing from the scope of the invention, it is intended that all matter contained in the above description or shown in the accompanying drawings shall be interpreted as illustrative and not in a limiting sense.

Referring to FIGS. 3-6B, a bone fixation device of the present invention is indicated generally 10 in FIG. 3. Device 10 serves two functions. First, the device is used to repair a bone fracture Fx in a bone. Second, and importantly, device 10 helps prevent a subsequent displacement of the reduced fracture of the bone. For example, the bone can be a person's (and particularly a woman's) femur WF. In a femur, a center section CS of the bone is of an elongate, generally cylindrically shape; while the respective ends E of the femur (only one of which is shown in the drawings) are on the order of four times larger then the center portion of the bone.

In repairing bone WF after a fracture, a rod 12 is inserted into the soft, center tissue of the bone. The rod is inserted into one end of the bone and extends longitudinally through the bone as shown in FIG. 3. The length of the rod is such that when inserted into the bone it bridges across the fracture Fx shown in FIG. 3. This allows the rod to be used to align the portions CS and E of the bone on either side of the fracture. It will be understood that while the fracture shown in FIG. 3 extends generally horizontally of the bone, many fractures extend sideways of the bone, vertically, as well as diagonally.

Next, a plurality of screws 14a, 14b are threaded into the bone, transversely of the bone and through transverse openings 15 in rod 12. The screws are spaced apart from each other along the length of the bone and are inserted into the bone on both sides of the fracture. In FIG. 3, shorter screws 14a are threaded into the bone above the fracture along the narrower center section CS of the femur. Longer screws 14b are inserted into the bone adjacent the larger end E thereof. As shown in the drawings, the length of the respective screws is such that they extend substantially across the transverse width of the bone. When in place, the screws retain rod 12 in place. Well-known surgical techniques are used to insert the screws through the openings in the rod and are not described.

To prevent future fracture of the bone as a result of aging and the concomitant bone loss which occurs in many people, a thin walled plate 16 is fitted against the outside of the bone. The length of plate 16 is such that when fitted against the side of the bone, a lower end 16a of the plate is adjacent the end of the bone, while the upper end 16b of the plate extends along the length of the bone past the location of the fracture. Each plate has a plurality of longitudinally spaced openings 18a-18e. While five such openings are shown in the drawings, those skilled in the art will understand the plate may have more or fewer openings. However, there is at least one opening 18 in the plate on each side of the fracture when the plate is fitted in place.

To facilitate insertions of the screws 14, and insure their proper alignment, the openings 18 in plate 16 can be of different shapes. Thus, in FIG. 4, one of the openings, opening 18b is referred to as a “cauliflower” opening because of its appearance. Other openings, 18a and 18c, are shown to be elongate openings. Other of the openings, 18a, 18d, 18e, are round openings. These various opening shapes makes it easier to align the tip end of the screw with the outer surface of the bone and so makes it easier to start threading the screw into the bone. Importantly, the diameter or width of each of the openings 18 in plate 16 is smaller in diameter than a head of the screws 14a or 14b used to attach the plate to the bone. If, over time, the bone loses mass, plate 16 will prevent the head of the screws from sinking into the spongy tissue Y of the bone and creating stresses which, as previously discussed, can cause the displacement of the reduced fracture. To further help prevent such a displacement, a washer 20 is used with each screw. Each washer is positioned between the head of a screw inserted through one of the openings 18 and plate 16, so to better distribute the forces produced by the screw when threaded into the bone. The shape of the washer conforms to that of the side of the plate at the location of the opening 18 in the plate.

In FIG. 3, two plates 16 are shown positioned against opposite sides of the bone. Use of the plate shown on the left side of the drawing figure is optional. If both plates are used, each plate is contoured to match the outer surface of the side of the bone against which it abuts. Further, washers 20 are used with both ends of the screws 14b.

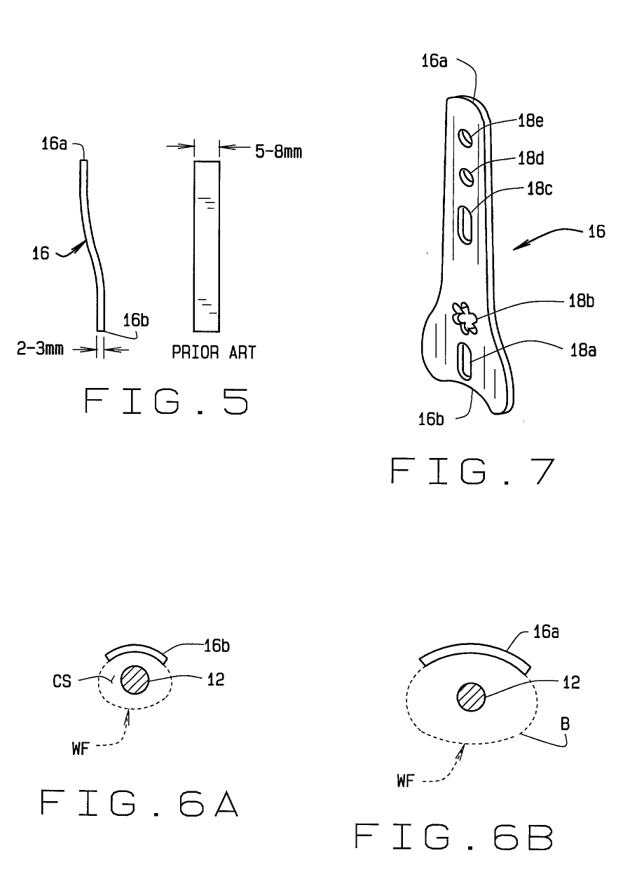

As particularly shown in FIGS. 6A, 6B, and 7, each plate 16 is a curved plate whose curvature conforms to the contour of the bone throughout the length of the plate. The plate decreases in width along its length. Accordingly, the plate is wider at its end 16a adjacent the end of the bone and narrows in width to its end 16b as it extends along the length of the bone. Further, lower end 16a of the plate bows or bends outwardly so to conform to outer surface of the enlarged, lobulated end of the bone. A major feature of the invention is that plate 16 is a very thin plate, particularly when compared to plates used in conventional bone repair surgery. In FIG. 5, plate 16 is shown to be on the order of 2-3 mm thick. As also shown in the drawing, conventional plates are on the order of 5-8 mm thick, or typically at least twice as thick as plate 16. One advantage of the thinness of the plate is that a surgeon using the plate in repairing a bone fracture can manually conform the plate about the outside of the bone, so that it closely conforms to the shape of the outer surface of the bone when the surgeon fits the plate into place.

The advantage in using a very thin wall plate 16 in bone repair surgery is twofold: First, the plate provides a support structure for use in repairing the fracture. It is comparable to someone placing the palm of their hand against the side of the bone to help hold the two parts of the bone together. The ability of the surgeon to form the plate about the bone aids in this regard. Second, the plate provides a barrier to future migration of the screws inserted into the bone through the plate and into the bone. Thus, even if the person subsequently loses bone mass, the screws cannot sink into the bone and result in displacement of the reduced fracture. Since bone fractures in older adults are often more difficult to repair, and take longer to heal, this is of a particular benefit to them.

Finally, those skilled in the art will appreciate that the plate 16 may come in several sizes. Each plate is a very thin walled plate conformable to the outer surface of the patient's bone as the surgeon fits the plate into place. The surgeon selects the appropriate size plate during the surgery and installs it in the manner described above.

In view of the above, it will be seen that the several objects and advantages of the present invention have been achieved and other advantageous results have been obtained.

Having thus described the invention, what is claimed and desired to be secured by Letters Patent is:

1. A bone fixation device for repairing a bone fracture and helping prevent a subsequent displacement of a reduced fracture comprising:
a rod inserted into the bone, longitudinally of the bone, the length of the rod being such as to extend across the fracture and align the portions of the bone on either side of the fracture;
a plurality of screws threaded into the bone and inserted through spaced openings in the rod so to attach the rod to the bone on both sides of the fracture; and,
a thin walled plate fitted against the outside of the bone adjacent one end thereof, the plate being a contoured plate contoured to match the outer surface of the bone, the plate having a plurality of spaced openings therein through which the screws are threaded into the bone, the size of the openings being smaller in diameter than a head of the screws being used so that, over time, as the bone loses mass due to aging, the plate prevents the screws from sinking into the bone and creating stresses which can cause a displacement of the reduced fracture.

2. The bone fixation device of claim 1 further including a second thin walled plate fitted against the outside of the bone opposite the first said plate, the second plate also being a contoured plate contoured to match the outer surface of the bone and having a plurality of spaced openings therein through which screws are threaded into the bone, the size of the openings again being smaller in diameter than a head of the screws being used.

3. The bone fixation device of claim 1 in which the thinness of a plate is such that a surgeon using the plate in repairing a bone fracture can form the plate about the outside of the bone so that the plate conforms to the outer contour of the bone.

4. The bone fixation device of claim 3 wherein each plate has a thickness of less than 3 mm.

5. The bone fixation device of claim 1 further including a washer used with each screw threaded into the bone through one of the openings in the plate, the washer being fitted between the head of the screw and the plate to distribute the forces created when a screw is inserted into the bone.

6. The bone fixation device of claim 3 in which the plate is a curved plate whose curvature conforms to the contour of the bone throughout the length of the plate.

7. The bone fixation device of claim 6 in which the length of the plate is such that when fitted against the side of the bone, the plate extends from the end of the bone past the location of the fracture, the spacing of the openings in the plate being such that at least one opening in the plate is on each side of the fracture when the plate is in place.

8. The bone fixation device of claim 7 in which the plate increases in width along its length, the plate being wider at its end adjacent the end of the bone and narrowing in width along its portion extending along the length of the bone

9. The bone fixation device of claim 1 wherein the plate is available in several sizes, each plate being a thin walled plate conformable to the outer surface of the patient's bone for a surgeon to select the appropriate size plate during a bone repair surgery.

10. A method of repairing a bone fracture and helping prevent a subsequent displacement of the reduced fracture comprising:
inserting a rod into the bone, longitudinally of the bone, the length of the rod being such as to extend across the fracture and align the portions of the bone on either side of the fracture;
threading a plurality of screws into the bone and through spaced openings in the rod, the screws being inserted transversely of the bone along the length of the bone and on both sides of the fracture; and,
fitting a thin walled plate against the outside of the bone adjacent one end thereof, the plate being a contoured plate contoured to match the outer surface of the bone and having a plurality of spaced openings therein through which the screws are threaded into the bone, the size of the openings being smaller in diameter than a head of the screws threaded into the bone whereby, over time, as the bone loses mass due to aging, the plate prevents the screws from sinking into the bone and creating stresses which can cause a displacement of the reduced fracture.

11. The method of claim 10 further including fitting a second thin walled plate against the outside of the bone opposite the first said plate, the second plate also being a contoured plate contoured to match the outer surface of the bone and having a plurality of spaced openings therein through which screws are threaded into the bone, the size of the openings again being smaller in diameter than a head of the screws being used.

12. The method of claim 11 each plate has a thickness of 2-3 mm.

13. The method of claim 10 further including using a washer with each screw threaded into the bone through one of the openings in the plate, the washer being positioned between the head of the screw and the plate to distribute the force created by the screw being inserted into the bone.

14. The method of claim 10 wherein the thinness of the plate is such that a surgeon using the plate in repairing a bone fracture can form the plate about the outside of the bone so the plate conforms to the outer contour of the bone.

15. The method of claim 14 in which the length of the plate is such that when fitted against the side of the bone, the plate extends from the end of the bone past the location of the fracture.

16. The method of claim 10 further including selecting an appropriately sized plate from a plurality of different size thin wall plates for use in repairing the fracture.

17. A bone fixation device for repairing a bone fracture and helping prevent a subsequent displacement of the reduced fracture comprising:
a rod inserted into the bone, longitudinally of the bone, the length of the rod being such as to extend across the fracture and align the portions of the bone on either side of the fracture;
a plurality of screws threaded into the bone and inserted through spaced openings in the rod so to attach the rod to the bone on both sides of the fracture; and,
a thin walled plate fitted against the outside of the bone adjacent one end thereof, the plate being a contoured plate contoured to match the outer surface of the bone, the length of the plate being such that when fitted against the side of the bone the plate extends from the end of the bone past the location of the fracture, the plate having a plurality of spaced openings therein through which screws are threaded into the bone with at least one opening in the plate being on each side of the fracture when the plate is in place, the size of the openings being smaller in diameter than a head of the screws being used so that, over time, as the bone loses mass due to aging, the plate prevents the screws from sinking into the bone and creating stresses which can cause a displacement of the reduced fracture.

18. The bone fixation device of claim 17 wherein the thinness of the plate is such that a surgeon using the plate in repairing a bone fracture can form the plate about the outside of the bone so that the plate conforms to the outer contour of the bone.

19. The bone fixation device of claim 17 further including a second thin walled plate which is fitted against the outside of the bone opposite the first said plate, the second plate also being a contoured plate contoured to match the outer surface of the bone and having a plurality of spaced openings therein through which screws are threaded into the bone, the size of the openings again being smaller in diameter than a head of the screws being used.

20. The bone fixation device of claim 18 further including a washer for each screw threaded into the bone through one of the openings in the plate, the washer distributing the forces created by insertion of the screw into the bone.

21. The bone fixation device of claim 18 in which each plate is a curved plate whose curvature conforms to the contour of the bone throughout the length of the plate, and the plate increases in width along its length, the plate being wider at its end adjacent the end of the bone and narrowing in width along its portion extending along the length of the bone.

22. The bone fixation device of claim 21 wherein the plate is available in several sizes, each plate being is a thin walled plate conformable to the outer surface of the patient's bone for a surgeon to select the appropriate size plate during a bone repair surgery.

23. The bone fixation device of claim 17 for repairing a bone fracture to a person's femur.
